(19) United States
(12) Patent Application Publication
(10) Pub. No.: US 2026/0158415 A1
(43) Pub. Date: Jun. 11, 2026

(54) METHODS FOR IDENTIFICATION AND QUANTITATION OF ABALOPARATIDE AND RELATED PEPTIDE IMPURITIES

(71) Applicant: Radius Health, Inc., Boston, MA (US)

(72) Inventor: Qun Jiang, Wayne, PA (US)

(73) Assignee: Radius Health, Inc., Boston, MA (US)

(21) Appl. No.: 19/400,661

(22) Filed: Nov. 25, 2025

Related U.S. Application Data
(63) Continuation of application No. PCT/IB2024/055317, filed on May 30, 2024.
(60) Provisional application No. 63/505,561, filed on Jun. 1, 2023.

(51) Int. Cl. B01D 15/16; B01D 15/42; G01N 30/02; G01N 30/04; G01N 30/34; G01N 30/38; G01N 33/15

(52) U.S. Cl. CPC B01D 15/166; B01D 15/426; G01N 30/04; G01N 30/34; G01N 30/38; G01N 33/15; G01N 2030/022

(57) ABSTRACT

Provided herein are methods of analyzing samples from batches of abaloparatide for abaloparatide and abaloparatide-related peptide impurities.

### CROSS-REFERENCE TO RELATED APPLICATIONS

This application is a continuation of International Application No. PCT/IB2024/055317, filed May 30, 2024, which claims priority to U.S. Provisional Patent Application No. 63/505,561, filed Jun. 1, 2023, each of which are herein incorporated by reference in their entirety for all purposes.

### TECHNICAL FIELD

The present disclosure relates to improved methods for the separation and quantitation of abaloparatide and abaloparatide-related impurities in samples which include abaloparatide.

### SEQUENCE LISTING

The instant application contains a sequence listing which has been submitted in XML format via Patent Center and is hereby incorporated by reference in its entirety. Said XML format copy, created on Nov. 25, 2025, is named SL1280US and is 2.21 kilobytes in size.

### BACKGROUND

Abaloparatide is a synthetic 34 amino acid peptide analog of human parathyroid hormone-related peptide, hPTHrP(1-34) having 76% homology to hPTHrP(1-34) and 41% homology to human parathyroid hormone (hPTH(1-34)). Abaloparatide has a molecular formula of C₁₇₄H₃₀₀N₅₆O₄₉, a molecular weight of 3961 daltons, and the amino acid sequence:

Ala¹-Val-Ser-Glu-His⁵-Gln-Leu-Leu-His-Asp¹⁰-Lys-Gly-Lys-Ser-lle¹⁵-Gln-Asp-Leu-Arg-Arg²⁰-Arg-Glu-Leu-Leu-Glu²⁵-Lys-Leu-Leu-Aib-Lys³⁰-Leu-His-Thr-Ala³⁴-NH₂ (SEQ. ID 1).

The structure and preparation of abaloparatide has been previously disclosed in, for example, U.S. Pat. No. 5,969,095, which is hereby incorporated by reference. A product containing abaloparatide, formulated for subcutaneous injection and used for treatment of osteoporosis in postmenopausal women, is available under the brand name TYMLOS, marketed by Radius Health Inc.

Batches of abaloparatide active pharmaceutical ingredient (API) may include varying amounts of peptide impurities related to abaloparatide, such as truncated peptides, acetylated peptides, peptides comprising a cyclized amino acid residue, and isomeric peptides. Further, upon storage, for example, in a formulated abaloparatide drug product, degradation impurities may form over time, and which may be the same or different from the peptide impurities related to abaloparatide initially present (e.g., in the initial API batch, or an initially formulated abaloparatide drug product).

In order to determine the suitability of abaloparatide API for manufacture of abaloparatide drug product, and to determine the suitability of abaloparatide drug product for administration, the levels of these abaloparatide-related impurities must be quantified and established as below particular thresholds per US Food and Drug Administration (FDA) regulations. Accordingly, it is desirable in the art to provide methods to efficiently and reproducibly separate and accurately quantify abaloparatide and each of the related impurities which may be present in samples of abaloparatide API and/or drug product.

### SUMMARY

The present technology is generally directed to methods for the separation and quantitation of abaloparatide and abaloparatide-related impurities in samples from batches comprising abaloparatide, such as abaloparatide API, initially formulated abaloparatide drug product, and formulated abaloparatide drug product following storage under a variety of conditions.

As discussed above, it is important to quantify abaloparatide-related impurities in various abaloparatide-containing batches to meet regulatory requirements. While the presence of small amounts of such impurities in TYMLOS have not been associated with any adverse event or toxicity, there is a desire to minimize the presence of such impurities to avoid the potential for e.g., immunogenic responses. Further, the beta-Asp¹⁰ impurity has been demonstrated to have a potency of only about 10% of that of abaloparatide. Accordingly, it is desirable to provide simple, accurate, and reproducible methods for separation and quantitation of abaloparatide-related impurities in samples comprising abaloparatide.

The methods according to the present disclosure provide a number of advantages over previous methods, including improved level of quantitation of abaloparatide related impurities, simplified mobile phase, extended column life, improved separation of abaloparatide related impurities, and the potential for determination of multiple abaloparatide related impurities in a single method. For example, as disclosed in U.S. Pat. No. 10,996,208, incorporated herein by reference, a chromatographic method utilizing trifluoroacetic acid in the mobile phase provided good separation between cyclo-Asp¹⁰ and abaloparatide, but inadequate separation between beta-Asp¹⁰ and abaloparatide. Conversely, a prior chromatographic method, which utilized ammonium phosphate buffer in the mobile phase, provided good separation between beta-Asp¹⁰ and abaloparatide, but inadequate separation between cyclo-Asp¹⁰ and abaloparatide. In contrast, the present disclosure conveniently provides single methods which may be utilized to separate abaloparatide, beta-Asp¹⁰, and cyclo-Asp¹⁰. Further, the methods are robust, easily implemented, compatible with existing conventional ultra-high performance chromatography (UPLC) instrumentation, and allow for extended column life.

In one aspect is provided a chromatographic method of analyzing a sample of abaloparatide, the method comprising:
(i) providing a sample comprising abaloparatide;
(ii) introducing the sample to an ultra-performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluant comprising a first mobile phase comprising aqueous perchloric acid and a second mobile phase which is acetonitrile, thereby eluting the abaloparatide and any beta-Asp¹⁰ abaloparatide from the immobilized stationary phase in the eluant;
(iv) detecting the abaloparatide present in the eluant using a detector; and
(v) quantifying the abaloparatide present in the sample.

In some embodiments, the first mobile phase is an aqueous solution containing from about 0.05 to about 0.2 percent by weight of perchloric acid. In some embodiments, the first mobile phase is an aqueous solution containing about 0.09 to about 0.13 percent by weight of perchloric acid.

In some embodiments, the C18-modified particles are inorganic/organic hybrid particles. In some embodiments, the C18-modified particles are spherical particles having an average particle diameter in a range from about 1.5 to about 2 microns, and an average pore size in a range from about 100 to about 150 Å.

In some embodiments, the eluent has a flow rate between about 0.1 and about 1.0 mL/min.

In some embodiments, flowing the sample through the immobilized stationary phase is performed using gradient elution, wherein the gradient elution comprises:
providing an initial ratio of the first mobile phase to the second mobile phase of about 75:25;
decreasing the initial ratio to a second ratio of the first mobile phase to the second mobile phase of about 70:30 over a period of time of about 50 minutes;
maintaining the second ratio for about 2.5 minutes;
decreasing the second ratio to a third ratio of the first mobile phase to the second mobile phase of about 30:70 over a period of time of about 0.1 minutes; and
maintaining the third ratio for about 2 minutes.

In some embodiments, the column is maintained at a temperature in a range from about 15° C. to about 30° C.

In some embodiments, the detector is selected from the group consisting of a UV/VIS detector, PDA detector, fluorescence detector, mass spectrometer, refractive index detector, evaporative light scattering detector, a charged aerosol detector, or a combination thereof.

In some embodiments, the sample further comprises beta-Asp10 abaloparatide, the method further comprising:
in step (iii), eluting the beta-Asp10 abaloparatide from the immobilized stationary phase in the eluant;
in step (iv), detecting the beta-Asp10 abaloparatide present in the eluant using a detector; and
in step (v), quantifying the beta-Asp10 abaloparatide present in the sample.

In some embodiments, detecting the beta-Asp10 abaloparatide comprises detecting the absence of and/or determining the absence of beta-Asp10 abaloparatide.

In some embodiments, the sample is from a batch of abaloparatide drug substance.

In some embodiments, the method comprises determining the sample comprises an amount of beta-Asp10 abaloparatide which is ≤0.5% by weight, based on the total weight of the drug substance.

In some embodiments, the method further comprises qualifying the batch of abaloparatide drug substance as suitable for manufacture of a formulated abaloparatide drug product. In some embodiments, qualifying the batch comprises creating and approving a batch record, filing a submission with the FDA certifying the batch, or both.

In some embodiments, the method further comprises formulating the abaloparatide drug substance into an abaloparatide drug product suitable for subcutaneous administration.

In some embodiments, the method comprises determining the sample comprises an amount of beta-Asp10 abaloparatide which is >0.5% by weight, based on the total weight of the drug substance, and wherein the method comprises conducting further purification of the batch of abaloparatide drug substance, reworking the batch of abaloparatide drug substance, or both.

In some embodiments, the method further comprises formulating the further purified or reworked batch of abaloparatide drug substance into an abaloparatide drug product suitable for subcutaneous administration.

In some embodiments, the sample is from a batch of formulated abaloparatide drug product. In some embodiments, the batch of formulated abaloparatide drug product is a batch of an initially prepared, formulated abaloparatide drug product.

In some embodiments, quantifying the beta-Asp¹⁰ abaloparatide present in the sample comprises determining the sample comprises an amount of beta-Asp¹⁰ which is ≤0.5% by weight based on the total weight of abaloparatide present in the batch of the formulated abaloparatide drug product.

In some embodiments, the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, the method further comprises determining the suitability for administration to a subject of the batch of stored, formulated abaloparatide drug product, the determining comprising ascertaining that the sample comprises an amount of beta-Asp10 abaloparatide which is ≤5% by weight based on the total weight of abaloparatide in the batch of stored, formulated abaloparatide drug product.

In some embodiments, the sample further comprises cyclo-Asp¹⁰ abaloparatide, the method further comprising:
in step (iii), eluting the cyclo-Asp¹⁰ abaloparatide from the immobilized stationary phase in the eluant;
in step (iv), detecting the cyclo-Asp¹⁰ abaloparatide present in the eluant using a detector; and
in step (v), quantifying the cyclo-Asp¹⁰ abaloparatide present in the sample.

In some embodiments, the sample is from a batch of abaloparatide drug substance.

In some embodiments, the method comprises determining the sample comprises an amount of cyclo-Asp¹⁰ abaloparatide which is ≤0.5% by weight, based on the weight of abaloparatide.

In some embodiments, the sample is from a batch of formulated abaloparatide drug product. In some embodiments, the batch of formulated abaloparatide drug product is a batch of an initially prepared, formulated abaloparatide drug product.

In some embodiments, quantifying the cyclo-Asp¹⁰ abaloparatide present in the sample comprises determining the sample comprises an amount of cyclo-Asp¹⁰ which is ≤0.5% by weight based on the weight of abaloparatide in the batch of the formulated abaloparatide drug product.

In some embodiments, the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, the method further comprises determining the suitability for administration to a subject of the batch of stored, formulated abaloparatide drug product, the determining comprising ascertaining that the sample comprises an amount of cyclo-Asp¹⁰ abaloparatide which is ≤3% by weight based on the total weight of abaloparatide in the batch of stored, formulated abaloparatide drug product.

In another aspect is provided a method of providing abaloparatide in a form substantially free of isomeric and degradation impurities, the method comprising:
(i) providing a sample from a batch of abaloparatide;
(ii) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluent comprising a first mobile phase comprising aqueous perchloric acid and a second mobile phase which is acetonitrile, thereby eluting components of the sample from the immobilized stationary phase in the eluant;
(iv) detecting and quantifying the isomeric and degradation impurities in the sample using a detector;
(v quantifying the amount of beta-Asp10 abaloparatide present relative to abaloparatide;
(vi) ascertaining that the amount beta-Asp10 abaloparatide is not more than 1% by weight based on the weight of abaloparatide; and
(vii) approving release of the batch of abaloparatide.

In another aspect is provided a method of manufacturing an abaloparatide drug product suitable for subcutaneous administration, the method comprising:
(i) analyzing a sample from a batch of abaloparatide drug product, wherein analyzing comprises:
   a) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
   b) flowing the sample through the immobilized stationary phase for a period of time with an eluent comprising a first mobile phase comprising aqueous perchloric acid and a second mobile phase which is acetonitrile, thereby eluting components of the sample from the immobilized stationary phase in the eluant;
   c) detecting and quantifying any isomeric and degradation impurities present in the sample using a detector;
ii) determining the abaloparatide is substantially free of isomeric and degradation impurities; and
iii) approving the release of the batch of abaloparatide drug product.

In some embodiments, the batch of formulated abaloparatide drug product is a batch of an initially prepared, formulated abaloparatide drug product.

In some embodiments, determining the batch of abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 1% by weight based on the weight of abaloparatide; and
ascertaining that the amount cyclo-Asp¹⁰ abaloparatide is not more than 0.5% by weight based on the weight of abaloparatide.

In some embodiments, the batch of abaloparatide drug product has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, determining the stored batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 5% by weight based on the weight of abaloparatide; and
ascertaining that the amount cyclo-Asp¹⁰ abaloparatide is not more than 3% by weight based on the weight of abaloparatide.

In another aspect is provided a chromatographic method of analyzing a sample of abaloparatide, the method comprising:
(i) providing a sample comprising abaloparatide;
(ii) introducing the sample to an ultra-performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting the abaloparatide from the immobilized stationary phase in the eluant;
(iv) detecting the abaloparatide present in the eluant using a detector; and
(v) quantifying the abaloparatide present in the sample.

In some embodiments, the first mobile phase is an aqueous solution containing from about 0.05 to about 0.2 percent by weight of formic acid and from about 0.05 to about 0.1% by weight of heptafluorobutyric acid.

In some embodiments, the second mobile phase is an acetonitrile solution containing from about 0.05 to about 0.2 percent by weight of formic acid and from about 0.05 to about 0.1% by weight of heptafluorobutyric acid.

In some embodiments, the C18-modified particles are inorganic/organic hybrid particles.

In some embodiments, the C18-modified particles are spherical particles having an average particle diameter in a range from about 1.5 to about 2 microns, and an average pore size in a range from about 100 to about 150 Å.

In some embodiments, the eluent has a flow rate between about 0.1 and about 1.0 mL/min.

In some embodiments, flowing the sample through the immobilized stationary phase is performed using gradient elution, wherein the gradient elution comprises:
providing an initial ratio of the first mobile phase to the second mobile phase of about 70:30;
decreasing the initial ratio to a second ratio of the first mobile phase to the second mobile phase of about 60:40 over a period of time of about 25 minutes;
decreasing the second ratio to a third ratio of the first mobile phase to the second mobile phase of about 10:90 over a period of time of about 3 minutes; and
maintaining the third ratio for about 2 minutes.

In some embodiments, the column is maintained at a temperature in a range from about 15° C. to about 30° C.

In some embodiments, the system further comprises an inline static mixer upstream from the column.

In some embodiments, the detector is selected from the group consisting of a UV/VIS detector, PDA detector, fluorescence detector, mass spectrometer, refractive index detector, evaporative light scattering detector, a charged aerosol detector, or a combination thereof. In some embodiments, the detector comprises a UV/VIS detector and a mass spectrometer.

In some embodiments, the sample further comprises beta-Asp¹⁰ abaloparatide, the method further comprising:
in step (iii), eluting the beta-Asp¹⁰ abaloparatide from the immobilized stationary phase in the eluant;
in step (iv), detecting the beta-Asp¹⁰ abaloparatide present in the eluant using a detector; and
in step (v), quantifying the beta-Asp¹⁰ abaloparatide present in the sample.

In some embodiments, the sample is from a batch of abaloparatide drug substance.

In some embodiments, the method comprises determining the sample comprises an amount of beta-Asp10 abaloparatide which is ≤0.5% by weight, based on the total weight of the drug substance.

In some embodiments, the method further comprises qualifying the batch of abaloparatide drug substance as suitable for manufacture of a formulated abaloparatide drug product.

In some embodiments, qualifying the batch comprises creating and approving a batch record, filing a submission with the FDA certifying the batch, or both.

In some embodiments, the method further comprises formulating the abaloparatide drug substance into an abaloparatide drug product suitable for subcutaneous administration.

In some embodiments, the method comprises determining the sample comprises an amount of beta-Asp10 abaloparatide which is >0.5% by weight, based on the total weight of the drug substance, and wherein the method comprises conducting further purification of the batch of abaloparatide drug substance, reworking the batch of abaloparatide drug substance, or both.

In some embodiments, the method further comprises formulating the further purified or reworked batch of abaloparatide drug substance into an abaloparatide drug product suitable for subcutaneous administration.

In some embodiments, the sample is from a batch of formulated abaloparatide drug product.

In some embodiments, the batch of formulated abaloparatide drug product is a batch of an initially prepared, formulated abaloparatide drug product.

In some embodiments, quantifying the beta-Asp¹⁰ abaloparatide present in the sample comprises determining the sample comprises an amount of beta-Asp¹⁰ which is ≤0.5% by weight based on the total weight of abaloparatide present in the batch of the formulated abaloparatide drug product.

In some embodiments, the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, the method further comprises determining the suitability for administration to a subject of the batch of stored, formulated abaloparatide drug product, the determining comprising ascertaining that the sample comprises an amount of beta-Asp¹⁰ abaloparatide which is ≤5% by weight based on the total weight of abaloparatide in the batch of stored, formulated abaloparatide drug product.

In some embodiments, the sample further comprises cyclo-Asp¹⁰ abaloparatide, the method further comprising:
in step (iii), eluting the cyclo-Asp¹⁰ abaloparatide from the immobilized stationary phase in the eluant;
in step (iv), detecting the cyclo-Asp¹⁰ abaloparatide present in the eluant using a detector; and
in step (v), quantifying the cyclo-Asp¹⁰ abaloparatide present in the sample.

In some embodiments, the sample is from a batch of abaloparatide drug substance.

In some embodiments, the method comprises determining the sample comprises an amount of cyclo-Asp¹⁰ abaloparatide which is ≤0.5% by weight, based on the weight of abaloparatide.

In some embodiments, the sample is from a batch of formulated abaloparatide drug product.

In some embodiments, the batch of formulated abaloparatide drug product is a batch of an initially prepared, formulated abaloparatide drug product.

In some embodiments, quantifying the cyclo-Asp¹⁰ abaloparatide present in the sample comprises determining the sample comprises an amount of cyclo-Asp¹⁰ which is ≤0.5% by weight based on the weight of abaloparatide in the batch of the formulated abaloparatide drug product.

In some embodiments, the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, the method further comprises determining the suitability for administration to a subject of the batch of stored, formulated abaloparatide drug product, the determining comprising ascertaining that the sample comprises an amount of cyclo-Asp¹⁰ abaloparatide which is ≤3% by weight based on the total weight of abaloparatide in the batch of stored, formulated abaloparatide drug product.

In another aspect is provided a method of providing abaloparatide in a form substantially free of beta-Asp¹⁰ abaloparatide, the method comprising:
(i) providing a sample from a batch of abaloparatide;
(ii) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting components of the sample from the immobilized stationary phase in the eluant;
(iv) detecting abaloparatide and beta-Asp¹⁰ abaloparatide in the sample using a detector;
(v) quantifying the amount of beta-Asp¹⁰ abaloparatide present relative to abaloparatide;
(vi) ascertaining that the amount beta-Asp¹⁰ abaloparatide is not more than 1% by weight based on the weight of abaloparatide; and
(vii) approving release of the batch of abaloparatide.

In yet another aspect is provided a method of manufacturing an abaloparatide drug product suitable for subcutaneous administration, the method comprising:
(i) analyzing a sample from a batch of abaloparatide drug product, wherein analyzing comprises:
   a) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
   b) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting components of the sample from the immobilized stationary phase in the eluant;
   c) detecting and quantifying any isomeric and degradation impurities present in the sample using a detector;
ii) determining the abaloparatide is substantially free of isomeric and degradation impurities; and
iii) approving the release of the batch of abaloparatide.

In some embodiments, the batch of abaloparatide drug product is a batch of an initially prepared, formulated abaloparatide drug product.

In some embodiments, determining the batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 1% by weight based on the weight of abaloparatide; and
ascertaining that the amount cyclo-Asp¹⁰ abaloparatide is not more than 0.5% by weight based on the weight of abaloparatide.

In some embodiments, the batch of abaloparatide drug product is a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

In some embodiments, determining the stored batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 5% by weight based on the weight of abaloparatide; and
ascertaining that the amount cyclo-Asp¹⁰ abaloparatide is not more than 3% by weight based on the weight of abaloparatide.

### BRIEF DESCRIPTION OF THE DRAWINGS

In order to provide an understanding of aspects of the technology, reference is made to the appended drawings, which are not necessarily drawn to scale. The drawings are exemplary only and should not be construed as limiting the technology. The disclosure described herein is illustrated by way of example and not by way of limitation in the accompanying drawings.

FIG. 1 provides the structural formula of a fragment of abaloparatide indicating the aspartic acid residue at position 10 (Asp¹⁰) and provides the structural formula of a fragment of an isomeric abaloparatide impurity having the residue beta-Asp¹⁰ (β-Asp¹⁰) generated by a rearrangement of the Asp¹⁰ residue.

FIG. 2 provides the structural formula of a fragment of abaloparatide indicating the aspartic acid residue at position 10 (Asp¹⁰) and provides the structural formula of a fragment of an abaloparatide impurity having the residue cyclo-Asp¹⁰, generated by imidization of the Asp¹⁰ residue.

FIG. 3 depicts an exemplary chromatographic separation of abaloparatide and beta-Asp¹⁰ abaloparatide in a resolution sample (abaloparatide spiked with a known concentration of beta-Asp¹⁰ abaloparatide).

FIG. 4A depicts an exemplary chromatographic separation of abaloparatide and beta-Asp¹⁰ abaloparatide in an aged sample of abaloparatide drug product from a stability study.

FIG. 4B is an enlarged view of the chromatographic separation depicted in FIG. 4A.

FIG. 5 depicts an exemplary chromatographic separation of abaloparatide and beta-Asp¹⁰ abaloparatide in a resolution sample (abaloparatide spiked with a known concentration of beta-Asp¹⁰ abaloparatide).

### DETAILED DESCRIPTION

Before describing several example embodiments of the technology, it is to be understood that the technology is not limited to the details of construction or process steps set forth in the following description. The technology is capable of other embodiments and of being practiced or being carried out in various ways.

### Definitions

With respect to the terms used in this disclosure, the following definitions are provided. This application will use the following terms as defined below unless the context of the text in which the term appears requires a different meaning.

The term “about” used throughout this specification is used to describe and account for small fluctuations. For example, the term “about” can refer to less than or equal to ±5%, such as less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

The term “abaloparatide” as used herein refers to [Glu^{22,25}, Leu^{23,28,31}, Aib²⁹, Lys^{26,30}]hPTHrP(1-34)NH₂) (Ala-Val-Ser-Glu-His-Gln-Leu-Leu-His-Asp-Lys-Gly-Lys-Ser-Ile-Gln-Asp-Leu-Arg-Arg-Arg-Glu-Leu-Leu-Glu-Lys-Leu-Leu-Aib-Lys-Leu-His-Thr-Ala-NH₂, SEQ ID NO:1), a peptide analog of PTHrP(1-34). Each of the 34 amino acids in abaloparatide are alpha amino acids. Aib is 2-aminoisobutyric acid, also known as α-aminoisobutyric acid or dimethylglycine.

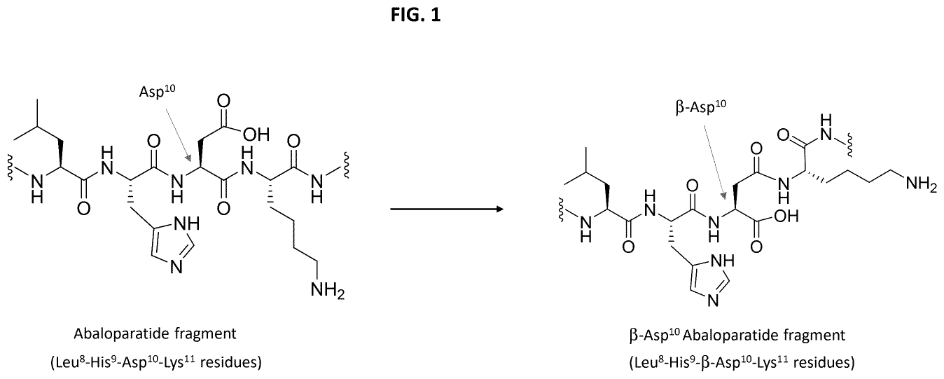

The terms “beta-Asp-abaloparatide,” “beta-Asp¹⁰,” “beta isomer,” and “(beta-Asp¹⁰) abaloparatide” as used herein refer to an isomer of abaloparatide in which the Asp at position 10 (Asp¹⁰) has been isomerized to beta-Asp: [β-Asp¹⁰, Glu^{22,25}, Leu^{23,28,31}, Aib²⁹, Lys^{26,30}]hPTHrP(1-34) NH₂) (Ala-Val-Ser-Glu-His-Gln-Leu-Leu-His-β-Asp-Lys-Gly-Lys-Ser-Ile-Gln-Asp-Leu-Arg-Arg-Arg-Glu-Leu-Leu-Glu-Lys-Leu-Leu-Aib-Lys-Leu-His-Thr-Ala-NH₂, SEQ ID NO:2). FIG. 1 shows a comparison of an abaloparatide fragment (residues 8-11) and the corresponding fragment of (beta-Asp10) abaloparatide.

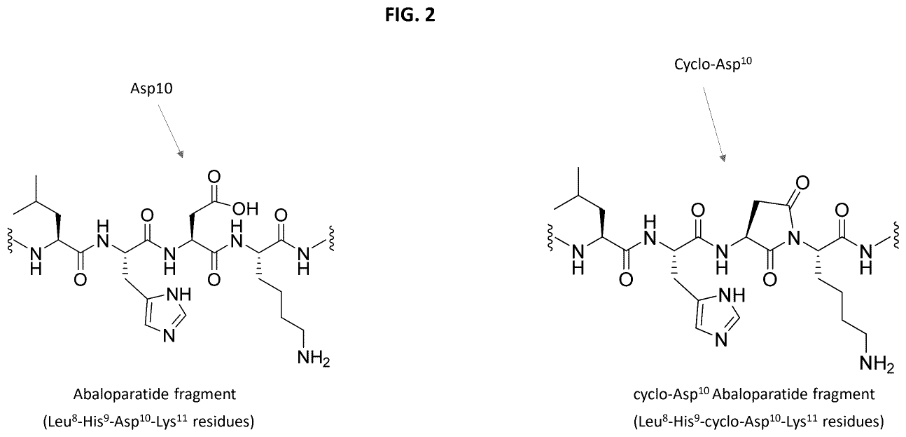

The terms “cyclo-Asp10 abaloparatide” as used herein refer to an isomer of abaloparatide in which the Asp at position 10 (Asp¹⁰) has been cyclized to form an imide. FIG. 2 shows a comparison of an abaloparatide fragment (residues 8-11) and the corresponding fragment of (cyclo-Asp¹⁰) abaloparatide.

The term “Active Pharmaceutical Ingredient” or “API” as used herein refers to a substance (e.g., abaloparatide) intended to be used in the manufacture of a drug (medicinal) product and that, when used in the production of a drug, becomes an active ingredient of the drug product.

The term “abaloparatide API” as used herein refers to abaloparatide that has undergone final manufacture and purification of the peptide but has not yet been formulated into a drug product (e.g., in an aqueous vehicle suitable for drug delivery). In certain embodiments, the abaloparatide API contains only abaloparatide, or, i.e., there are no significant additional or added components.

The term “drug product” as used herein refers to a finished dose form, e.g., a solution suitable for subcutaneous administration, that contains an API and optionally other inactive ingredients (e.g., water, buffer, and/or other excipients).

The term “abaloparatide drug product” as used herein refers to a product for administration to a subject in which abaloparatide API has been formulated in an aqueous vehicle suitable for drug delivery.

The term “chromatography” refers to a separation method for isolating one or more compounds found in a mixture. The compounds are normally present in a sample. This disclosure uses the term “sample” broadly to represent any mixture which an individual may desire to analyze. The term “mixture” is used in the sense of a fluid containing one or more dissolved compounds (e.g., abaloparatide and abaloparatide-related peptide impurities). Chromatography is a differential migration process. Compounds in a mixture traverse a chromatographic column at different rates, leading to their separation. The migration occurs by convection of a fluid phase, referred to as the mobile phase, in relationship to a packed bed of particles or a porous monolith structure, referred to as the stationary phase.

High Performance Liquid Chromatography (HPLC) is a chromatography technique used to separate, identify, and quantify each component in a mixture. HPLC relies on pumps to pass a pressurized liquid solvent (mobile phase) through a column filled with a sorbent (stationary phase), separating the components in a sample. Each component in the sample interacts slightly differently with the sorbent, causing different flow rates for the different components and leading to the separation of the components as they flow out of the column. HPLC is normally performed using a column having a packed bed of sorbent particles through which the mobile phase will flow. The column is placed in fluid communication with a pump and a sample injector. The sample is loaded onto the column under pressure by the sample injector and the sample components and mobile phase are pushed through the column by the pump. The column is placed in fluid communication with a detector, which can detect the change in the nature of the mobile phase as the mobile phase exits the column. The detector will register and record these changes as a plot, referred to as a chromatogram, which is used to determine the presence or absence of a particular component in the sample, and, in some embodiments, the concentration thereof. Examples of detectors used for HPLC are, without limitation, refractive index detectors, UV detectors, light-scattering detectors, and mass spectrometers.

Ultra-Performance Liquid Chromatography (UPLC) is a variant of HPLC using smaller sorbent particle size and higher operating pressure to dramatically increase resolution, speed and sensitivity for separations relative to HPLC. UPLC typically utilizes a combination of a sorbent having a particle size of less than about 2 μm and a fluid pressure in the 6000-15000 psi range (conventional HPLC uses 3-5 μm particle sizes and operates between 2000 and 4000 psi). This technology further allows for faster flow rates while maintaining separation efficiency, thereby increasing throughput.

Embodiments of the present disclosure are now described in detail as methods for performing chromatographic separations such as HPLC and UPLC with the understanding that such methods are exemplary methods. Such methods constitute what the inventors now believe to be the best mode of practicing the technology. Those skilled in the art will recognize that such methods are capable of modification and alteration.

### Chromatographic Methods

Provided herein are chromatographic methods of analyzing a sample comprising abaloparatide and optionally further comprising beta-Asp¹⁰ abaloparatide. The methods generally comprise: providing the sample; introducing the sample to an chromatography system comprising a column having an interior for accepting a stationary phase, and an immobilized stationary phase within said interior of the column; flowing the sample through the immobilized stationary phase for a period of time with an eluent; individually eluting the abaloparatide and beta-Asp¹⁰ abaloparatide from the immobilized stationary phase in the eluant; detecting the abaloparatide and beta-Asp¹⁰ abaloparatide present in the eluant using a detector; and quantifying the abaloparatide and beta-Asp¹⁰ abaloparatide present in the sample. In some embodiments, the method further comprises separating and quantitating additional abaloparatide-related impurities, including but not limited to cyclo-Asp¹⁰ abaloparatide. Accordingly, the methods may be useful in analyzing various abaloparatide-containing samples, such as abaloparatide API and formulated abaloparatide, such as initially prepared or stored formulated abaloparatide drug product. Each of the components and stages of the disclosed methods are described further herein below.

### Sample

The methods disclosed herein generally comprise providing a sample which includes abaloparatide, and may include additional abaloparatide related peptides including, but not limited to, beta-Asp¹⁰ abaloparatide. The sample to be analyzed may be from a batch of abaloparatide API, or may be from a batch of formulated abaloparatide, either before or after the formulated abaloparatide has been introduced to a device for administration (e.g., a subcutaneous injection pen). Further, the sample may be from initially prepared abaloparatide API or formulated abaloparatide, or after storage of the material.

In some embodiments, the sample is obtained from a batch of abaloparatide API. Abaloparatide, when initially prepared, may include certain impurities produced during manufacture. These impurities may include abaloparatide related peptide impurities such as the isomeric impurity beta-Asp¹⁰ abaloparatide, truncated peptides, acetylated peptides, and peptides comprising a cyclized residue (e.g., cyclo-beta-Asp¹⁰ abaloparatide). The presence of such impurities is considered undesirable, as they are either inactive or much less active than abaloparatide with respect to therapeutic effect. Further, the US Food and Drug Administration (FDA) imposes limitations on the content of impurities in all drugs in order to minimize the potential for toxicity and/or immunogenicity which may be associated with impurities. Accordingly, the FDA requires manufacturers of drug substances and drug products to identify, characterize, and quantitate impurities, keep records of such analyses, and further requires certain submissions with respect to such analyses to assure the efficacy and safety of drugs.

Generally, the presence of abaloparatide related peptide impurities in abaloparatide is low, as formation of such impurities is controlled during the manufacturing process, and purification steps remove such impurities during or at the end of the manufacturing process. Nevertheless, certain impurities may still be present in an initial batch, and therefore, batches of abaloparatide API require analysis to confirm impurities are below threshold levels in order to qualify a batch as suitable for further formulation. Further, as previously disclosed (see, e.g., U.S. Pat. No. 10,996,208 to Williams et al.), under certain conditions, abaloparatide can undergo an intramolecular rearrangement that results in formation of the beta-Asp¹⁰ isomer of abaloparatide as shown in FIG. 1. The rate of formation is dependent on multiple factors, including storage condition and temperature, but the impurity generally increases in concentration over time. Accordingly, it is important to analyze stored batches of abaloparatide API to ensure the quantity of beta-Asp¹⁰ abaloparatide remains below the threshold level. As discussed above, the presence of this relatively inactive impurity may serve to diminish the efficacy of abaloparatide products comprising beta-Asp¹⁰ abaloparatide.

Another abaloparatide-related peptide impurity which may form over time and/or be initially present in a batch of abaloparatide API is cyclo-Asp¹⁰ abaloparatide (FIG. 2). Accordingly, in some embodiments, the sample further comprises cyclo-Asp¹⁰ abaloparatide. Without wishing to be bound by theory, it is believed that storage of the abaloparatide API in the solid state may contribute to formation of this isomeric impurity over time by allowing cyclization of the free aspartic acid carboxyl group with the aspartic acid amide group in the abaloparatide backbone, forming an imide with loss of water.

The source of the sample may vary. For example, the present method is suitable for analyzing samples from various abaloparatide containing products, such as batches of abaloparatide which are in process or have been purified (e.g., abaloparatide API), formulated abaloparatide (e.g., prior to or after filling syringes with the abaloparatide drug product), or abaloparatide drug product after various periods of storage.

In some embodiments, the sample is abaloparatide drug substance. In some embodiments, the abaloparatide drug substance comprises an amount of beta-Asp10 abaloparatide which is ≤1% by weight, based on the total weight of the drug substance. In some embodiments, the abaloparatide drug substance comprises an amount of beta-Asp10 abaloparatide which is ≤0.5% by weight, based on the total weight of the drug substance.

In some embodiments, the abaloparatide drug substance comprises an amount of cyclo-Asp¹⁰ abaloparatide which is ≤2% by weight, based on the total weight of the drug substance.

In some embodiments, the sample is obtained from formulated abaloparatide (i.e., abaloparatide drug product). Any impurities initially present in the abaloparatide API will by default be present in the formulated product. Further, upon storage, these impurities may increase in concentration over time, or new impurities may form. In some embodiments, the primary impurity formed is beta-Asp10 abaloparatide. In some embodiments, the abaloparatide drug product is formulated for subcutaneous administration. In some embodiments, the abaloparatide drug product formulation has a pH between 4.6-5.5. In some embodiments, the pH is about 5.1 or about 5.2. In some embodiments, the abaloparatide drug product comprises a buffer. In some embodiments, the buffer is an acetate buffer, for example acetic acid or sodium acetate, or is a phosphate buffer, for example potassium phosphate. In In some embodiments, the buffer is in a concentration range sufficient to provide the desired level of buffer capacity, for example 0.1 mM to 60 mM, 0.5 to 50 mM, 1 to 10 mM, 4 to 8 mM, or about 6 mM. In some embodiments, the abaloparatide drug product further comprises an antimicrobial agent, for example a compound with a phenolic group such as chlorocresol or phenol, at a concentration sufficient to provide anti-microbial effect. In certain embodiments, the antimicrobial agent may be phenol at a concentration of ≤8.0 mg, for example≤5.0 mg/mL or about 5.0 mg/mL.

In some embodiments, the sample is from a batch of formulated abaloparatide drug product. In some embodiments, the formulated abaloparatide drug product comprises an amount of beta-Asp¹⁰ which is ≤0.5% by weight based on the total weight of abaloparatide present in the batch of the formulated abaloparatide drug product.

In some embodiments, the formulated abaloparatide drug product has been stored for up to 23 months at 2-8° C., has been stored for up to 23 months at 2-8° C. followed by a month at 20-25° C., or has been stored for up to 35 months at 2-8° C. followed by a month at 20-25° C.

### Column

The methods disclosed herein generally comprise introducing the sample to a liquid chromatography system comprising a column having an interior for accepting a stationary phase, and an immobilized stationary phase within said interior of the column. In some embodiments, the liquid chromatography system is an ultra-performance chromatography (UPLC) system. For use in UPLC, generally, the stationary phase will be immobilized in a housing having a wall defining a chamber, for example, a column having an interior for accepting the stationary phase. Such columns will have a length and a diameter. In some embodiments, the length of the column is about 300 mm. In some embodiments, the length of the column is about 150 mm. In some embodiments, the length of the column is less than about 300 mm, less than about 150 mm, less than about 100 mm, or less than about 50 mm. In some embodiments, the length of the column is about 50 mm, about 30 mm, about 20 mm, or about 10 mm.

In some embodiments, the column has a bore size of about 4.6 mm inside diameter (i.d.). In some embodiments, the column has a bore size of greater than 4.6 mm i.d. In some embodiments, the column has a bore size of about 7.8 mm i.d. In some embodiments, the column has a bore size of greater than 7.8 mm i.d. In some embodiments, the column has a bore size of greater than about 4 mm i.d., greater than about 5 mm i.d., greater than about 6 mm i.d., or greater than about 7 mm i.d.

### Static Mixer

In some embodiments, the system further comprises positioned upstream of the column. It has been found according to the present disclosure that in some embodiments, the presence of such mixer greatly reduces system noise and enhances baseline separation. Accordingly, in some embodiments, the method further comprises flowing the first and second mobile phases into the inline mixer and allowing the two mobile phases to mix and form the eluant prior to flowing the eluant through the immobilized stationary phase. Suitable inline mixers are known to one of skill in the art. Examples of such inline mixers are inline static mixers available from Mott Corporation, Farmington, CT, USA (PerfectPeak®), and from Waters, Inc., Milford, MA, USA.

### Stationary Phase Material

The methods of the present disclosure utilize a stationary phase material enclosed in a column as described herein above. Such material can be composed of one or more particles, such as one or more spherical particles. The particles are generally spherical but can be any shape useful in chromatography.

The particles have a particle size or distribution of particle sizes. Particle size may be measured, e.g., using a Beckman Coulter Multisizer 3 instrument as follows. Particles are suspended homogeneously in a 5% lithium chloride methanol solution. A greater than 70,000 particle count may be run using a 30 μm aperture in the volume mode for each sample. Using the Coulter principle, volumes of particles are converted to diameter, where a particle diameter is the equivalent spherical diameter, which is the diameter of a sphere whose volume is identical to that of the particle. Particle size can also be determined by light microscopy.

The particles generally have a size distribution in which the average (mean) diameter is from about 1 to about 20 μm. In some embodiments, the particles have a diameter with a mean size distribution from about 1.5 μm to about 5 μm. In some embodiments, the particles have a size distribution in which the average diameter is about 1.7 μm. In some embodiments, the particles have a size distribution in which the average diameter is about 3 μm.

The particles are generally porous. The porous particles may comprise any suitable material. Suitable materials include, but are not limited to silica, inorganic/organic hybrid materials, and polymeric materials. In some embodiments, the porous particles comprise silica, an inorganic/organic hybrid material, or a polymer. In some embodiments, the porous particles comprise inorganic/organic hybrid materials.

In some embodiments, the stationary phase comprises silicon-based particles modified with a carbon component, such as a C4, C8, or C18 modified silica. In some embodiments, the stationary phase comprises C18 modified silica with a particle size in a range from about 1.5 to 2 μm and a pore size in a range from about 100 to about 150 Å. One particularly suitable example of commercial columns comprising such a stationary phase are ACQUITY® peptide CSH-C18 columns available from Waters Corp., Medford, MA, such as a 1.7 μm, 3.0×100 mm, 130 Å, ACQUITY® peptide CSH-C18 UPLC column. Another example of a suitable column is the ACQUITY CSH Phenyl-Hexyl Column 1.7 μm, 2.1×150 mm (Waters).

### Eluant

The methods disclosed herein comprises flowing the sample through an immobilized stationary phase for a period of time with an eluant. In certain specific embodiments, the eluant and, optionally the sample, are provided by a high-performance liquid chromatography (HPLC) system or an ultra-performance liquid chromatography (UPLC) system.

### First Mobile Phase

In some embodiments, the eluant comprises a first mobile phase comprising aqueous perchloric acid (HClO₄). Generally, the aqueous perchloric acid is a dilute solution prepared by diluting concentrated perchloric acid (e.g., about 70% perchloric acid by weight in water) with sufficient water to provide the desired concentration of perchloric acid. The concentration of perchloric acid present in the first mobile phase may vary. Surprisingly, according to the present disclosure, it was found that eluting the sample with an eluant comprising dilute aqueous perchloric acid provided enhanced resolution for separation and quantitation of beta-Asp¹⁰ in the presence of abaloparatide. In contrast, a previous method utilizing aqueous trifluoroacetic acid as the first mobile phase provided inadequate separation and quantitation of beta-Asp¹⁰ in the presence of abaloparatide, though it did allow separation and quantitation of cyclo-Asp¹⁰ in the presence of abaloparatide.

In some embodiments, the first mobile phase comprises aqueous perchloric acid wherein the quantity of perchloric acid present in the water is in a range from about 0.05 to about 0.2 percent by weight, such as from about 0.09 to about 0.13 percent perchloric acid by weight. In specific embodiments, the first mobile phase is 0.12 percent perchloric acid by weight.

In some embodiments, the eluant comprises a first mobile phase comprising aqueous heptafluorobutyric acid (HFBA). In some embodiments, the first mobile phase comprises aqueous HFBA and further comprises a carboxylic acid. In some embodiments, the carboxylic acid is formic acid, acetic acid, or trifluoroacetic acid. In some embodiments, the first mobile phase is aqueous formic acid and HFBA. Surprisingly, according to the present disclosure, it was found that eluting the sample with an eluant comprising water, formic acid, heptafluorobutyric acid, and water provided enhanced resolution for separation and quantitation of beta-Asp¹⁰ in the presence of abaloparatide. In contrast, a previous method utilizing aqueous trifluoroacetic acid in the first aqueous mobile phase provided inadequate separation and quantitation of beta-Asp¹⁰ in the presence of abaloparatide.

The concentration of formic and heptafluorobutyric acid present in the first mobile phase may vary. In some embodiments, the first mobile phase contains from about 0.05 to about 0.2 percent by weight of formic acid and from about 0.05 to about 0.1% by weight of HFBA in water.

### Second Mobile Phase

In some embodiments, the eluant comprises a second mobile phase which is an organic solvent. Organic solvents include, but are not limited to, methanol, ethanol, isopropanol, and acetonitrile. In some embodiments, the second mobile phase comprises or is methanol. In some embodiments, the second mobile phase comprises or is acetonitrile.

In some embodiments, the second mobile phase comprises acetonitrile and further comprises a carboxylic acid. In some embodiments, the second mobile phase comprises methanol and further comprises a carboxylic acid. In some embodiments, the carboxylic acid is formic acid, acetic acid, or trifluoroacetic acid. In some embodiments, the second mobile phase is acetonitrile including formic acid and HFBA. In some embodiments, the second mobile phase contains from about 0.05 to about 0.2 percent by weight of formic acid and from about 0.05 to about 0.1% by weight of HFBA in acetonitrile.

In some embodiments, the first mobile phase is aqueous formic acid and HFBA, and the second mobile phase is acetonitrile including formic acid and HFBA.

### Eluting

### Mobile Phase Ratio

The various components present in the sample are eluted from the stationary phase by flowing the eluant and sample through the stationary phase for a period of time. The relative amounts of the first and second mobile phases in the eluant may vary. The relative amounts of the first and second mobile phases in the eluant may be held constant (isocratic elution) or may vary over time (gradient elution), or a combination thereof.

In some embodiments, the disclosed methods comprise performing gradient elution. In some embodiments, performing gradient elution comprises providing an initial ratio of the first mobile phase to the second mobile phase; and decreasing the initial ratio to a second ratio of the first mobile phase to the second mobile phase over a period of time. Optionally, performing gradient elution further comprises maintaining one or more of the initial, the second and the third ratio for a period of time, independently selected for each occurrence. In some embodiments, the gradient elution comprises: providing an initial ratio of the first mobile phase to the second mobile phase of about 75:25; decreasing the initial ratio to a second ratio of the first mobile phase to the second mobile phase of about 70:30 over a period of time of about 50 minutes; maintaining the second ratio for about 2.5 minutes; decreasing the second ratio to a third ratio of the first mobile phase to the second mobile phase of about 30:70 over a period of time of about 0.1 minutes; and maintaining the third ratio for about 2 minutes. This non-limiting example embodiment of a gradient elution is illustrated in Table 1. This gradient is particularly suitable to the disclosed eluant comprising perchloric acid, water, and acetonitrile.

TABLE 1
Exemplary Gradient Conditions

| Time | Mobile Phase 1 | Mobile Phase 2 |
|---|---|---|
| (minutes) | (Aq. HClO₄), % | (CH₃CN), % |
| 0.0 | 75.5 | 24.5 |
| 48.1 | 70.3 | 29.7 |
| 50.6 | 70.3 | 29.7 |
| 50.7 | 30.0 | 70.0 |
| 52.7 | 30.0 | 70.0 |
| 52.8 | 75.5 | 24.5 |
| 60.0 | 75.5 | 24.5 |

In some embodiments, the gradient elution comprises: providing an initial ratio of the first mobile phase to the second mobile phase of about 70:30; decreasing the initial ratio to a second ratio of the first mobile phase to the second mobile phase of about 60:40 over a period of time of about 25 minutes; decreasing the second ratio to a third ratio of the first mobile phase to the second mobile phase of about 10:90 over a period of time of about 3 minutes; and maintaining the third ratio for about 2 minutes. This non-limiting example embodiment of a gradient elution is illustrated in Table 2. This gradient is particularly suitable to the disclosed eluant comprising formic acid, HFBA, water, and acetonitrile.

TABLE 2
Exemplary Gradient Conditions

| Time | Mobile Phase 1 | Mobile Phase 2 |
|---|---|---|
| (minutes) | (Aq. FA/HFBA), % | (CH₃CN/FA/HFBA), % |
| 0.0 | 71 | 29 |
| 2 | 69 | 31 |
| 4 | 69 | 31 |
| 25 | 60 | 40 |
| 28 | 10 | 90 |
| 30 | 10 | 90 |
| 30.1 | 71 | 29 |
| 40 | 71 | 29 |

### Flow Rate

The disclosed methods comprise flowing the eluant through the stationary phase. The eluant may be flowed at a variety of different flow rates, which may be determined by one of skill in the art based on scale, stationary phase particle size, difficulty of separation, and the like. In some embodiments, flowing the eluant through the immobilized stationary phase is performed at a flow rate from about 0.1 mL/min to about 3 mL/min. In some embodiments, the flow rate is less than 1 mL/min, such as from about 0.1, about 0.2, about 0.3, about 0.4, or about 0.5, to about 0.6, about 0.7, about 0.8, about 0.9, or about 1 mL/min. In some embodiments, the flow rate is about 0.3 mL/min. In some embodiments, the flow rate is about 0.25 mL/min.

### Temperature

The temperature at which the chromatography is performed (i.e., column temperature) may vary. In some embodiments, the column temperature is from about 15 to about 50° C., such as about 15, about 20, about 25, about 30, about 35, about 40, about 45, or about 50° C. In some embodiments, the column temperature is in a range from about 15° C. to about 30° C., such as about 15, about 20, about 25, or about 30° C. In some embodiments, the column temperature is about 20° C.

### Time

The time required for the separation will vary depending on many factors, but will generally be less than about 60 minutes, less than about 50 minutes, less than about 40 minutes, less than about 30 minutes, less than about 20 minutes, less than about 10 minutes, less than about 5 minutes, less than about 4 minutes, less than about 3 minutes, less than about 2 minutes, or less than about 1 minute. In particular, the time will be determined by the elution time of the component of interest, also referred to herein as retention time. In some embodiments, the retention time is reproducible from run to run.

### Detecting

The methods generally comprise detecting the presence or absence of a component (e.g., abaloparatide, beta-Asp¹⁰, cyclo-Asp¹⁰, or combinations thereof) in the sample. Many suitable options exist for methods of detection. In some embodiments, the detecting is performed using a refractive index detector, a UV detector, a light-scattering detector, a mass spectrometer, or combinations thereof. In specific embodiments, the detecting is performed using a UV detector. Numerous detectors are available; however, a specific detector is a Waters ACQUITY® UPLC® Tunable UV Detector (Waters Corporation, Milford, Mass., USA). In some embodiments, the detector comprises or further comprises a mass spectrometer. Particularly suitable for use with a mass spectrometry detector are the disclosed methods wherein the mobile phases comprise heptafluorobutyric acid.

As described above, the source of the sample analyzed according to the disclosed methods may vary. In some embodiments, a batch from which the sample is taken, and accordingly, the sample itself, will be substantially free of beta-Asp¹⁰ abaloparatide, meaning the amount present is below the limit of detection or quantification. Accordingly, in some embodiments, detecting the presence of beta-Asp¹⁰ abaloparatide comprises detecting an absence of beta-Asp¹⁰ abaloparatide, determining the absence of beta-Asp¹⁰ abaloparatide, or both. Likewise, the amount of cyclo-Asp¹⁰ abaloparatide present in a given sample may vary. Accordingly, in some embodiments, detecting the presence of cyclo-Asp¹⁰ abaloparatide comprises detecting an absence of cyclo-Asp¹⁰ abaloparatide, determining the absence of cyclo-Asp¹⁰ abaloparatide, or both.

### Quantifying

In some embodiments, the methods further comprise quantifying the amount of abaloparatide and/or abaloparatide-related impurities (e.g., beta-Asp¹⁰ abaloparatide and/or cyclo-Asp¹⁰ abaloparatide) present in a sample. The method used to quantify the abaloparatide and abaloparatide-related impurities present will vary based on the method of detection utilized, but generally involves peak integration of the chromatogram according to known methods, combined with comparison to reference standards containing known amounts of abaloparatide and/or abaloparatide-related impurities.

In general, and unless stated otherwise, when percentages and percent ranges and percent limits are given for abaloparatide, impurities, or degradants, the values are determined from UPLC integration ratios where such UPLC integration procedures are calibrated in accord with standard techniques known to one of ordinary skill in the art. A percentage of peptide impurities or degradants and/or abaloparatide are derived from the ratio of the particular impurity and/or degradant and/or abaloparatide divided by the total area of the total peptide content in the chromatogram (and multiplied by 100%), and the sum total of all peptide content from an abaloparatide-containing API or aqueous formulation should equal about 100%.

### Qualifying

In some embodiments, the methods further comprise qualifying a batch of abaloparatide (e.g., drug substance or formulated drug product). Qualifying generally comprises determining that the amount of abaloparatide and abaloparatide-related impurities fall within predetermined parameters. Qualifying a batch may comprise further activities such as documentation, including but not limited to creating and approving a batch record, filing a submission with the FDA to certify the batch, or both. In some embodiments, the method comprises qualifying a batch of abaloparatide drug substance as suitable for manufacture of a formulated abaloparatide drug product. In some embodiments, qualifying the batch comprises first determining the batch comprises an amount of beta-Asp10 abaloparatide which is ≤0.5% by weight, based on the total weight of the drug substance. In some embodiments, qualifying the batch comprises first determining the batch comprises an amount of cyclo-Asp¹⁰ abaloparatide which is ≤0.5% by weight, based on the weight of abaloparatide.

### Formulating

In some embodiments, the methods further comprise formulating the abaloparatide drug substance into an abaloparatide drug product suitable for subcutaneous administration. In some embodiments, formulating the abaloparatide drug substance into an abaloparatide drug product suitable for subcutaneous administration first comprises determining the batch comprises an amount of beta-Asp10 abaloparatide which is ≤0.5% by weight, based on the total weight of the drug substance. In some embodiments, the method comprises quantifying the cyclo-Asp10 abaloparatide present in the sample wherein quantifying comprises determining the sample comprises an amount of cyclo-Asp¹⁰ abaloparatide which is ≤0.5% by weight based on the weight of abaloparatide in the batch of the formulated abaloparatide drug product.

In some embodiments, a batch may be determined to comprise an amount of beta-Asp10 abaloparatide which is >0.5% by weight, based on the total weight of the drug substance. In such embodiments, the method comprises conducting further purification of the batch of abaloparatide drug substance, reworking the batch of abaloparatide drug substance, or both. In some embodiments, the further purified or reworked batch of abaloparatide drug substance is then formulated into an abaloparatide drug product suitable for subcutaneous administration. In some embodiments, a batch may be determined to comprise an amount of cyclo-Asp¹⁰ abaloparatide which is >0.5% by weight, based on the total weight of the drug substance. In such embodiments, the method comprises conducting further purification of the batch of abaloparatide drug substance, reworking the batch of abaloparatide drug substance, or both. In some embodiments, the further purified or reworked batch of abaloparatide drug substance is then formulated into an abaloparatide drug product suitable for subcutaneous administration.

### Suitability

In some embodiments, the methods further comprise determining the suitability for administration to a subject of a batch of formulated abaloparatide drug product, such as after a period of storage. Determining suitability generally comprises determining that the amount of abaloparatide and abaloparatide-related impurities fall within predetermined parameters. In some embodiments, determining the suitability for administration comprises ascertaining that the sample comprises an amount of beta-Asp10 abaloparatide which is ≤5% by weight based on the total weight of abaloparatide in a batch of stored, formulated abaloparatide drug product.

In some embodiments, determining the suitability for administration comprises or further comprises ascertaining that the sample comprises an amount of cyclo-Asp10 abaloparatide which is ≤3% by weight based on the total weight of abaloparatide in the batch of stored, formulated abaloparatide drug product.

In some embodiments, the sample is from a batch of formulated abaloparatide drug product that has been stored for a period of time at one or more temperatures. In some embodiments, the batch has been stored for up to 23 months at a temperature in a range from about 2° C. to about 8° C., such as from about 1, about 2, about 3, about 6, or about 12 months, to about 18, or about 23 months. In some embodiments, the batch has been stored for up to 23 months at a temperature in a range from about 2° C. to about 8° C., followed by a month at a temperature in a range from about 20° C. to about 25° C. In some embodiments, the batch has been stored for up to 35 months at a temperature in a range from about 2° C. to about 8° C., such as from about 1, about 2, about 3, about 6, or about 12 months, to about 18, about 24, about 30, or about 35 months. In some embodiments, the batch has been stored for up to 35 months at a temperature in a range from about 2° C. to about 8° C., followed by a month at a temperature in a range from about 20° C. to about 25° C.

### Method of Providing Abaloparatide in a Form Substantially Free of Isomeric and Degradation Impurities

Provided herein are methods of providing abaloparatide in a form substantially free of isomeric and degradation impurities. The methods comprise: providing an abaloparatide sample from a batch of abaloparatide; introducing the sample to an ultra-high performance chromatography (UPLC) system comprising a column having an interior for accepting a stationary phase, and an immobilized stationary phase within said interior of the column, wherein the immobilized stationary phase comprises C18-modified particles; flowing the sample through the immobilized stationary phase for a period of time with an eluent comprising a first mobile phase comprising an aqueous acid and a second mobile phase comprising acetonitrile, thereby eluting components of the sample from the immobilized stationary phase in the eluant; detecting and quantifying the isomeric and degradation impurities in the sample using a detector; determining the abaloparatide is substantially free of isomeric and degradation impurities; and approving release of the abaloparatide. Each of the sample, UPLC system, column, stationary phase, flowing, period of time, eluent, mobile phases, eluting, and detecting are as described herein above.

In some embodiments, the first mobile phase is aqueous perchloric acid and the second mobile phase is acetonitrile. In some embodiments, the first mobile phase is aqueous formic acid and HFBA and the second mobile phase is formic acid and HFBA in acetonitrile.

In some embodiments, determining the abaloparatide is substantially free of isomeric and degradation impurities comprises quantifying an amount of beta-Asp10 abaloparatide present relative to abaloparatide; and ascertaining that the amount beta-Asp10 abaloparatide is not more than 1% by weight based on the weight of abaloparatide. In some embodiments, the determining comprises quantifying and ascertaining that the amount beta-Asp10 abaloparatide is less than about 1%, less than about 0.5%, less than about 0.1%, less than 0.05%, less than 0.01%, less than 0.001%, or even 0% by weight.

### Methods of Manufacturing

Provided herein are methods of manufacturing an abaloparatide drug product suitable for subcutaneous administration. The methods comprise:
(i) analyzing a sample from a batch of abaloparatide drug product, wherein analyzing comprises:
   a) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
   b) flowing the sample through the immobilized stationary phase for a period of time with an eluent comprising a first mobile phase comprising an aqueous acid and a second mobile phase comprising acetonitrile, thereby eluting components of the sample from the immobilized stationary phase in the eluant;
   c) detecting and quantifying any isomeric and degradation impurities present in the sample using a detector;
ii) determining the abaloparatide is substantially free of isomeric and degradation impurities; and
iii) approving the release of the batch of abaloparatide.

In some embodiments, the first mobile phase is aqueous perchloric acid and the second mobile phase is acetonitrile. In some embodiments, the first mobile phase is aqueous formic and heptafluorobutyric acid (HFBA) and the second mobile phase is formic acid and HFBA in acetonitrile.

In some embodiments, the sample is from a batch of an initially prepared, formulated abaloparatide drug product. In some embodiments, the batch of formulated abaloparatide drug product is a batch of an initially prepared, formulated abaloparatide drug product. In some embodiments, determining the batch of formulated abaloparatide drug product is substantially free of isomeric and degradation impurities comprises: quantifying an amount of beta-Asp¹⁰ abaloparatide present; quantifying an amount of cyclo-Asp¹⁰ abaloparatide present; ascertaining that the amount beta-Asp¹⁰ abaloparatide is not more than 1% by weight based on the weight of abaloparatide; and ascertaining that the amount cyclo-Asp¹⁰ abaloparatide is not more than 0.5% by weight based on the weight of abaloparatide.

In some embodiments, the sample is from a batch of formulated abaloparatide drug product that has been stored for a period of time at one or more temperatures. In some embodiments, the batch has been stored for up to 23 months at a temperature in a range from about 2° C. to about 8° C., such as from about 1, about 2, about 3, about 6, or about 12 months, to about 18, or about 23 months. In some embodiments, the batch has been stored for up to 23 months at a temperature in a range from about 2° C. to about 8° C., followed by a month at a temperature in a range from about 20° C. to about 25° C. In some embodiments, the batch has been stored for up to 35 months at a temperature in a range from about 2° C. to about 8° C., such as from about 1, about 2, about 3, about 6, or about 12 months, to about 18, about 24, about 30, or about 35 months. In some embodiments, the batch has been stored for up to 35 months at a temperature in a range from about 2° C. to about 8° C., followed by a month at a temperature in a range from about 20° C. to about 25° C.

In some embodiments, determining the stored batch of formulated abaloparatide drug product is substantially free of isomeric and degradation impurities comprises: quantifying an amount of beta-Asp¹⁰ abaloparatide present; quantifying an amount of cyclo-Asp¹⁰ abaloparatide present; ascertaining that the amount beta-Asp¹⁰ abaloparatide is not more than 5% by weight based on the weight of abaloparatide; and ascertaining that the amount cyclo-Asp¹⁰ abaloparatide is not more than 3% by weight based on the weight of abaloparatide.

### Methods of Providing Abaloparatide Substantially Free of Impurities

Further provided are methods of providing abaloparatide in a form substantially free of isomeric and degradation impurities, such as beta-Asp¹⁰ abaloparatide and cyclo-Asp¹⁰ abaloparatide. The methods generally comprise: providing an abaloparatide sample; introducing the sample to an ultra-high performance chromatography (UPLC) system comprising a column having an interior for accepting a stationary phase, and an immobilized stationary phase within said interior of the column, wherein the immobilized stationary phase comprises C18-modified particles; flowing the sample through the immobilized stationary phase for a period of time with an eluent comprising a first mobile phase comprising an aqueous acid and a second mobile phase comprising acetonitrile, thereby eluting components of the sample from the immobilized stationary phase in the eluant; detecting and quantifying the isomeric and degradation impurities in the sample using a detector; determining the abaloparatide is substantially free of isomeric and degradation impurities; and approving release of the abaloparatide. Each of the sample, UPLC system, column, stationary phase, flowing, period of time, eluent, mobile phases, eluting, detecting, quantifying, determining, and approving release are as described herein above.

In some embodiments, the first mobile phase is aqueous perchloric acid and the second mobile phase is acetonitrile. In some embodiments, the first mobile phase is aqueous formic acid and HFBA and the second mobile phase is formic and HFBA in acetonitrile.

In some embodiments, determining the abaloparatide is substantially free of isomeric and degradation impurities comprises quantifying an amount of beta-Asp¹⁰ abaloparatide present relative to abaloparatide; and ascertaining that the amount beta-Asp¹⁰ abaloparatide is not more than 1% by weight based on the weight of abaloparatide.

All methods described herein can be performed in any suitable order unless otherwise indicated herein or otherwise clearly contradicted by context. The use of any and all examples, or exemplary language (e.g., “such as”) provided herein, is intended merely to better illustrate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the technology. Thus, the appearances of phrases such as “‘in one or more embodiments,’” “‘in certain embodiments,” “in one embodiment” or “in an embodiment” in various places throughout this specification are not necessarily referring to the same embodiment of the technology. Furthermore, the particular features, structures, materials, or characteristics may be combined in any suitable manner in one or more embodiments. Any ranges cited herein are inclusive.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### EXEMPLIFICATION

The present invention may be further illustrated by the following non-limiting examples describing the methods.

### Example 1: General Method 1 for Identification of Peptide Impurities in Samples Containing Abaloparatide

This method is suitable for the determination of abaloparatide and related impurities in samples of abaloparatide API and abaloparatide injectable solution (abaloparatide drug product).

### Mobile Phase Preparation

Mobile phase A is a 0.119% solution of perchloric acid in water, prepared from a stock perchloric acid solution. Perchloric acid (70% w/w; 2.0 ml) was accurately pipetted into a 1000 mL volumetric flask containing approximately 500 mL of deionized water. The mixture was diluted to volume with water and mixed well to provide the stock solution (0.14% aqueous perchloric acid). A portion of the stock solution (850 mL) was diluted with water (150 mL) to prepare Mobile Phase A.

Mobile Phase B was 100% acetonitrile.

### Abaloparatide Standard Solutions

An abaloparatide Working Standard solution was prepared. Liquid Abaloparatide Reference Standard Solution (400 μL; ˜0.8 mg/mL) was pipetted into a suitable sample vial and diluted with deionized water to a concentration of 0.25 mg/mL.

An abaloparatide Sensitivity Standard solution was prepared. Working Standard Solution (100 μL) was pipetted into a 100 mL volumetric flask and diluted with deionized water to a concentration of 0.00025 mg/mL.

### Beta-Asp-10 Standard Solutions

A beta-Asp¹⁰ abaloparatide Working Standard solution was prepared. Beta-Asp¹⁰ abaloparatide (5.2 mg) was accurately weighed into a 25 mL volumetric flask, dissolved in deionized water, and diluted to volume to provide a 0.21 mg/mL standard solution.

A beta-Asp¹⁰ abaloparatide Resolution Standard was prepared. Abaloparatide API (12.5 mg) was accurately weighed into a 50 mL volumetric flask. The above beta-Asp-¹⁰ abaloparatide standard solution (3.0 mL) was pipetted into the same flask, dissolved in deionized water, and diluted to volume to provide a resolution standard solution containing 0.25 mg/mL abaloparatide and 0.0125 mg/mL beta-Asp¹⁰ abaloparatide. The level of quantitation of beta-Asp¹⁰ abaloparatide was determined to be 0.3%.

### Sample Preparation

Uniformity of Dosage Unit (UDU, 0.25 mg/mL)

Ten cartridges are randomly pulled for each batch. From each cartridge, a 0.625 mL sample is removed and diluted to 5 mL with deionized water.

Bulk Product/In-Process Testing (0.25 mg/mL)

Bulk sample (2.5 mL) is pipetted into a 20 mL flask and diluted up to volume with deionized water.

Assay and Impurity (0.25 mg/mL)

Ten cartridges are randomly pulled for each batch. From each cartridge, a 2.5 mL sample is removed and diluted to 20 mL with deionized water.

### Identification Sample Preparation

Abaloparatide API (12.5 mg) is weighed into a 50 mL volumetric flask, dissolved in deionized water and diluted up to volume with deionized water. From this solution, an aliquot is withdrawn and mixed with an equal volume of working standard.

### Injectable Solution

Equal volumes of the assay sample and the working standard solution are mixed.

### Instrumentation

The chromatography system used is an Acquity™ H-Class UPLC system (available from Waters Corporation, Milford, MA), running Empower Chromatography Data System (CDS) Software. The system includes a Quaternary Solvent Manager, Sample Manager FTN-H, Column Manager, and PDA Detector. The column is a CSH-C18, 1.7 μm, 3.0×100 mm, 130 Å UPLC column (Waters SKU: 186005301). Gradient elution is performed according to Table 3 using a flow rate of 0.30 mL/min. The column temperature is 20±2° C., the autosampler temperature is maintained at 8±2° C. The injection volume is 10 μL. The run time is 60 minutes, and the PDA detector wavelength is set to 214 nm.

TABLE 3
Gradient Conditions

| Time | Mobile Phase 1 | Mobile Phase 2 |
|---|---|---|
| (minutes) | (Aq. HClO₄), % | (CH₃CN), % |
| 0.0 | 75.5 | 24.5 |
| 48.1 | 70.3 | 29.7 |
| 50.6 | 70.3 | 29.7 |
| 50.7 | 30.0 | 70.0 |
| 52.7 | 30.0 | 70.0 |
| 52.8 | 75.5 | 24.5 |
| 60.0 | 75.5 | 24.5 |

### Example 2. Analysis of Resolution Sample

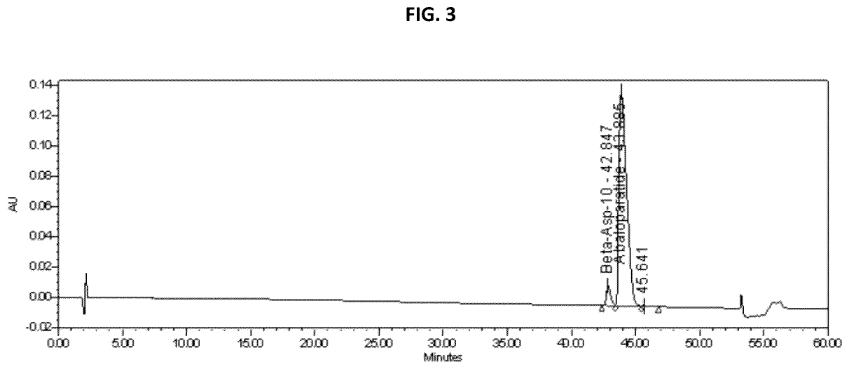

A sample (10 μL) of the standard resolution solution prepared as described in Example 1 (containing 0.25 mg/mL abaloparatide and 0.0125 mg/mL beta-Asp10 abaloparatide) was analyzed according to the method described in Example 1. An exemplary chromatogram is provided in FIG. 3, showing the sample contained 4.94% by area of beta-Asp10 abaloparatide and 94.83% by area of abaloparatide. This data demonstrates the method provided baseline separation of the two components in the standard, and accurately indicated the relative concentrations of the two components.

### Example 3. Analysis of Stability Sample

A sample of abaloparatide drug product from a 9-month stability study was analyzed according to the method described in Example 1. A sample of the aged abaloparatide formulated product (12.5 mg) was weighed into a 50 mL volumetric flask, dissolved in deionized water and diluted up to volume with deionized water. From this solution, an aliquot (10 μL) was withdrawn and analyzed using the system and conditions described in Example 1.

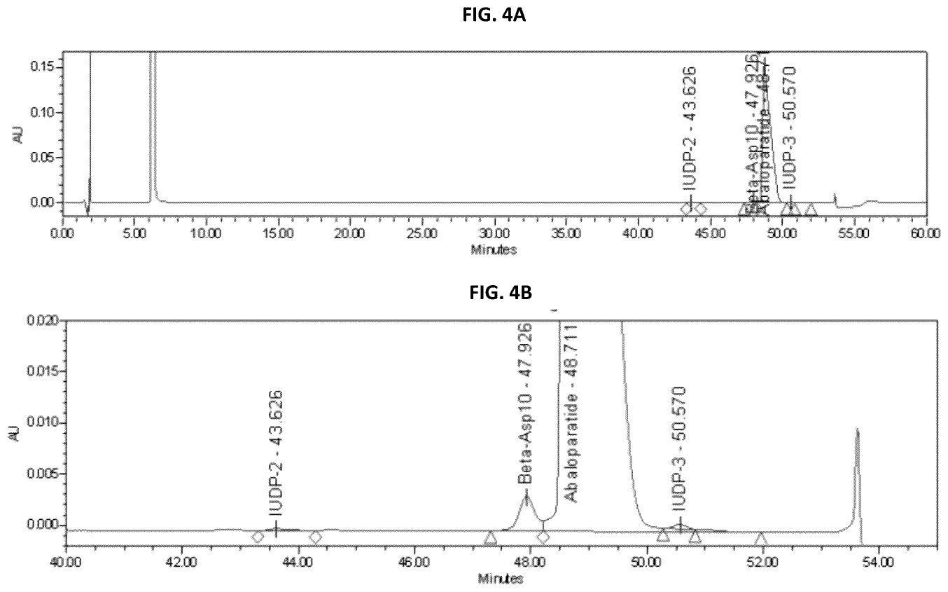

An exemplary chromatogram is provided in FIG. 4A, with an enlarged view relative to FIG. 4A provided as FIG. 4B. With reference to FIGS. 4A and 4B, the chromatograms show the sample contained 99.2% by area of abaloparatide and 1.14% by area of beta-Asp10 abaloparatide. This data demonstrates the method provided baseline separation of the two components in the sample and allowed quantitation of the components.

### Example 4: General Method 2 for Identification of Peptide Impurities in Samples Containing Abaloparatide

This method is suitable for the determination of abaloparatide and related impurities in samples of abaloparatide API and abaloparatide injectable solution (abaloparatide drug product).

### Mobile Phase Preparation

Mobile phase A is a solution of 0.1% formic acid and 0.075% heptafluorobutyric acid (HFBA) in water. Mobile Phase B was 0.1% formic acid and 0.075% HFBA in acetonitrile.

### Beta-Asp-10 Standard Solutions

A beta-Asp¹⁰ abaloparatide stock solution is prepared. Beta-Asp10 abaloparatide (5 mg) is accurately weighed into a 5 mL volumetric flask, dissolved in deionized water, and diluted to volume.

A beta-Asp¹⁰ abaloparatide Resolution Standard is prepared from beta-Asp10 abaloparatide and abaloparatide standard stock solutions. The Resolution Standard solution contains 0.95 mg/mL abaloparatide and 0.05 mg/mL beta-Asp10 abaloparatide.

### Instrumentation

The chromatography system used is an Acquity™ H-Class UPLC system (available from Waters Corporation, Milford, MA), running Empower Chromatography Data System (CDS) Software. The system includes a Quaternary Solvent Manager, Sample Manager FTN-H, Column Manager, PDA Detector, and a Mott PerfectPeak® inline static mixer. The column is an ACQUITY Peptide CSH-C18, 1.7 μm, 3.0×100 mm, 130 Å UPLC column (Waters SKU: 186006983). Gradient elution is performed according to Table 4 using a flow rate of 0.25 mL/min. The column temperature is 30±2° C., the autosampler temperature is maintained at 10±2° C. The injection volume is 15 μL. The run time is 40 minutes, and the PDA detector wavelength is set to 220 nm.

TABLE 4
Gradient Conditions

| Time | Mobile Phase 1 | Mobile Phase 2 |
|---|---|---|
| (minutes) | (Aq. FA/HFBA), % | (CH₃CN/FA/HFBA), % |
| 0.0 | 71 | 29 |
| 2 | 69 | 31 |
| 4 | 69 | 31 |
| 25 | 60 | 40 |
| 28 | 10 | 90 |
| 30 | 10 | 90 |
| 30.1 | 71 | 29 |
| 40 | 71 | 29 |

### Example 5. Analysis of Resolution Sample

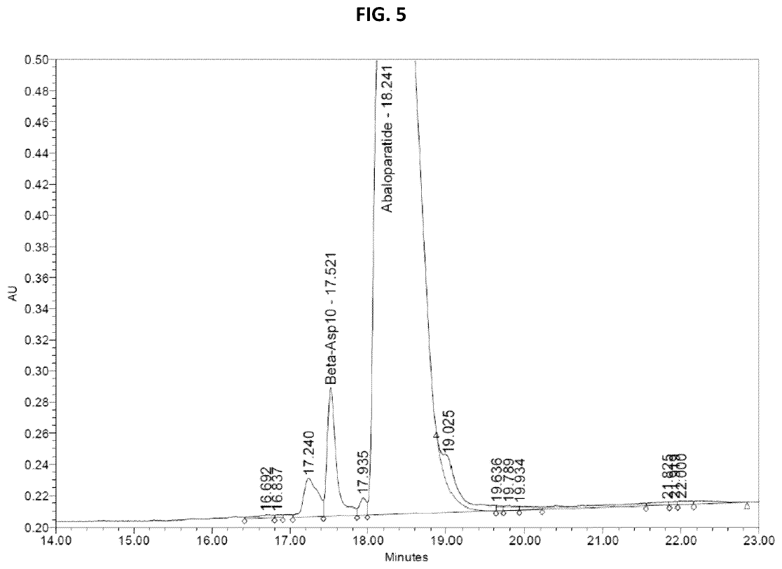

A sample (15 μL) of the standard resolution solution prepared as described in Example 4 was analyzed according to the method described in Example 4. An exemplary chromatogram is provided in FIG. 5. With reference to FIG. 5, the chromatogram shows baseline separation of abaloparatide and Beta-Asp10 abaloparatide, and further shows the separation of additional minor impurities which were not previously detected by other UPLC methods (e.g., aqueous perchloric acid and acetonitrile as in Example 1, and prior reported methods using aqueous TFA and acetonitrile). This data demonstrates the method provided baseline separation of the components in the standard, and accurately indicated the relative concentrations of the components. Notably, the inclusion of a static in line mixer significantly reduced the baseline noise, enhancing separation and quantification of the impurities.

What is claimed is:

1. A chromatographic method of analyzing a sample of abaloparatide, the method comprising:
(i) providing a sample comprising abaloparatide;
(ii) introducing the sample to an ultra-performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluant comprising a first mobile phase comprising aqueous perchloric acid and a second mobile phase which is acetonitrile, thereby eluting the abaloparatide from the immobilized stationary phase in the eluant;
(iv) detecting the abaloparatide present in the eluant using a detector; and
(v) quantifying the abaloparatide present in the sample.

2. The method of claim 1, wherein the first mobile phase is an aqueous solution containing from about 0.05 to about 0.2 percent by weight of perchloric acid.

3. The method of claim 1 or 2, wherein the first mobile phase is an aqueous solution containing about 0.09 to about 0.13 percent by weight of perchloric acid.

4. The method of any one of claims 1-3, wherein the C18-modified particles are inorganic/organic hybrid particles.

5. The method of any one of claims 1-4, wherein the C18-modified particles are spherical particles having an average particle diameter in a range from about 1.5 to about 2 microns, and an average pore size in a range from about 100 to about 150 Å.

6. The method of any one of claims 1-5, wherein the eluent has a flow rate between about 0.1 and about 1.0 mL/min.

7. The method of any one of claims 1-6, wherein flowing the sample through the immobilized stationary phase is performed using gradient elution, wherein the gradient elution comprises:
providing an initial ratio of the first mobile phase to the second mobile phase of about 75:25;
decreasing the initial ratio to a second ratio of the first mobile phase to the second mobile phase of about 70:30 over a period of time of about 50 minutes;
maintaining the second ratio for about 2.5 minutes;
decreasing the second ratio to a third ratio of the first mobile phase to the second mobile phase of about 30:70 over a period of time of about 0.1 minutes; and
maintaining the third ratio for about 2 minutes.

8. The method of any one of claims 1-7, wherein the column is maintained at a temperature in a range from about 15° C. to about 30° C.

9. The method of any one of claims 1-8, wherein the detector is selected from the group consisting of a UV/VIS detector, PDA detector, fluorescence detector, mass spectrometer, refractive index detector, evaporative light scattering detector, a charged aerosol detector, or a combination thereof.

10. The method of any one of claims 1-9, wherein the sample further comprises beta-Asp¹⁰ abaloparatide, the method further comprising:
in step (iii), eluting the beta-Asp¹⁰ abaloparatide from the immobilized stationary phase in the eluant;
in step (iv), detecting the beta-Asp¹⁰ abaloparatide present in the eluant using a detector; and
in step (v), quantifying the beta-Asp¹⁰ abaloparatide present in the sample.

11. The method of claim 10, wherein the sample is from a batch of abaloparatide drug substance.

12. The method of claim 11, wherein the method comprises determining the sample comprises an amount of beta-Asp¹⁰ abaloparatide which is ≤0.5% by weight, based on the total weight of the drug substance.

13. The method of claim 12, further comprising qualifying the batch of abaloparatide drug substance as suitable for manufacture of a formulated abaloparatide drug product.

14. The method of claim 12, wherein qualifying the batch comprises creating and approving a batch record, filing a submission with the FDA certifying the batch, or both.

15. The method of claim 12 or 13, further comprising formulating the abaloparatide drug substance into an abaloparatide drug product suitable for subcutaneous administration.

16. The method of claim 11, wherein the method comprises determining the sample comprises an amount of beta-Asp10 abaloparatide which is >0.5% by weight, based on the total weight of the drug substance, and wherein the method comprises conducting further purification of the batch of abaloparatide drug substance, reworking the batch of abaloparatide drug substance, or both.

17. The method of claim 16, further comprising formulating the further purified or reworked batch of abaloparatide drug substance into an abaloparatide drug product suitable for subcutaneous administration.

18. The method of claim 10, wherein the sample is from a batch of formulated abaloparatide drug product.

19. The method of claim 18, wherein the batch of formulated abaloparatide drug product is a batch of an initially prepared, formulated abaloparatide drug product.

20. The method of claim 19, wherein quantifying the beta-Asp¹⁰ abaloparatide present in the sample comprises determining the sample comprises an amount of beta-Asp¹⁰ which is ≤0.5% by weight based on the total weight of abaloparatide present in the batch of the formulated abaloparatide drug product.

21. The method of claim 10, wherein the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

22. The method of claim 21, further comprising determining the suitability for administration to a subject of the batch of stored, formulated abaloparatide drug product, the determining comprising ascertaining that the sample comprises an amount of beta-Asp¹⁰ abaloparatide which is ≤5% by weight based on the total weight of abaloparatide in the batch of stored, formulated abaloparatide drug product.

23. The method of any one of claims 1-22, wherein the sample further comprises cyclo-Asp¹⁰ abaloparatide, the method further comprising:
in step (iii), eluting the cyclo-Asp¹⁰ abaloparatide from the immobilized stationary phase in the eluant;
in step (iv), detecting the cyclo-Asp¹⁰ abaloparatide present in the eluant using a detector; and
in step (v), quantifying the cyclo-Asp¹⁰ abaloparatide present in the sample.

24. The method of claim 23, wherein the sample is from a batch of abaloparatide drug substance.

25. The method of claim 24, wherein the method comprises determining the sample comprises an amount of cyclo-Asp¹⁰ abaloparatide which is ≤0.5% by weight, based on the weight of abaloparatide.

26. The method of claim 23, wherein the sample is from a batch of formulated abaloparatide drug product.

27. The method of claim 26, wherein the batch of formulated abaloparatide drug product is a batch of an initially prepared, formulated abaloparatide drug product

28. The method of claim 27, wherein quantifying the cyclo-Asp¹⁰ abaloparatide present in the sample comprises determining the sample comprises an amount of cyclo-Asp¹⁰ which is ≤0.5% by weight based on the weight of abaloparatide in the batch of the formulated abaloparatide drug product.

29. The method of claim 23, wherein the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

30. The method of claim 29, further comprising determining the suitability for administration to a subject of the batch of stored, formulated abaloparatide drug product, the determining comprising ascertaining that the sample comprises an amount of cyclo-Asp¹⁰ abaloparatide which is ≤3% by weight based on the total weight of abaloparatide in the batch of stored, formulated abaloparatide drug product.

31. A method of providing abaloparatide in a form substantially free of beta-Asp¹⁰ abaloparatide, the method comprising:
(i) providing a sample from a batch of abaloparatide;
(ii) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluent comprising a first mobile phase comprising aqueous perchloric acid and a second mobile phase which is acetonitrile, thereby eluting components of the sample from the immobilized stationary phase in the eluant;
(iv) detecting abaloparatide and beta-Asp¹⁰ abaloparatide in the sample using a detector;
(v) quantifying the amount of beta-Asp¹⁰ abaloparatide present relative to abaloparatide;
(vi) ascertaining that the amount beta-Asp¹⁰ abaloparatide is not more than 1% by weight based on the weight of abaloparatide; and
(vii) approving release of the batch of abaloparatide.

32. A method of manufacturing an abaloparatide drug product suitable for subcutaneous administration, the method comprising:
(i) analyzing a sample from a batch of abaloparatide drug product, wherein analyzing comprises:
a) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
b) flowing the sample through the immobilized stationary phase for a period of time with an eluent comprising a first mobile phase comprising aqueous perchloric acid and a second mobile phase which is acetonitrile, thereby eluting components of the sample from the immobilized stationary phase in the eluant;
c) detecting and quantifying any isomeric and degradation impurities present in the sample using a detector;
ii) determining the abaloparatide is substantially free of isomeric and degradation impurities; and
iii) approving the release of the batch of abaloparatide.

33. The method of claim 32, wherein the batch of abaloparatide drug product is a batch of an initially prepared, formulated abaloparatide drug product.

34. The method of claim 33, wherein determining the batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 1% by weight based on the weight of abaloparatide; and
ascertaining that the amount cyclo-Asp¹⁰ abaloparatide is not more than 0.5% by weight based on the weight of abaloparatide.

35. The method of claim 32, wherein the batch of abaloparatide drug product is a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

36. The method of claim 35, wherein determining the stored batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 5% by weight based on the weight of abaloparatide; and
ascertaining that the amount cyclo-Asp¹⁰ abaloparatide is not more than 3% by weight based on the weight of abaloparatide.

37. A chromatographic method of analyzing a sample of abaloparatide, the method comprising:
(vi) providing a sample comprising abaloparatide;
(vii) introducing the sample to an ultra-performance chromatography (UPLC) system comprising a column having an interior for accepting a stationary phase, and an immobilized stationary phase within said interior of the column, wherein the immobilized stationary phase comprises C18-modified particles;
(viii) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting the abaloparatide from the immobilized stationary phase in the eluant;
(ix) detecting the abaloparatide present in the eluant using a detector; and
(x) quantifying the abaloparatide present in the sample.

38. The method of claim 37, wherein the first mobile phase is an aqueous solution containing from about 0.05 to about 0.2 percent by weight of formic acid and from about 0.05 to about 0.1% by weight of heptafluorobutyric acid.

39. The method of claim 37 or 38, wherein the second mobile phase is an acetonitrile solution containing from about 0.05 to about 0.2 percent by weight of formic acid and from about 0.05 to about 0.1% by weight of heptafluorobutyric acid.

40. The method of any one of claims 37-39, wherein the C18-modified particles are inorganic/organic hybrid particles.

41. The method of any one of claims 37-40, wherein the C18-modified particles are spherical particles having an average particle diameter in a range from about 1.5 to about 2 microns, and an average pore size in a range from about 100 to about 150 Å.

42. The method of any one of claims 37-41, wherein the eluent has a flow rate between about 0.1 and about 1.0 mL/min.

43. The method of any one of claims 37-42, wherein flowing the sample through the immobilized stationary phase is performed using gradient elution, wherein the gradient elution comprises:
providing an initial ratio of the first mobile phase to the second mobile phase of about 70:30;
decreasing the initial ratio to a second ratio of the first mobile phase to the second mobile phase of about 60:40 over a period of time of about 25 minutes;
decreasing the second ratio to a third ratio of the first mobile phase to the second mobile phase of about 10:90 over a period of time of about 3 minutes; and
maintaining the third ratio for about 2 minutes.

44. The method of any one of claims 37-43, wherein the column is maintained at a temperature in a range from about 15° C. to about 30° C.

45. The method of any one of claims 37-44, wherein the detector is selected from the group consisting of a UV/VIS detector, PDA detector, fluorescence detector, mass spectrometer, refractive index detector, evaporative light scattering detector, a charged aerosol detector, or a combination thereof.

46. The method of any one of claims 37-45, wherein the sample further comprises beta-Asp¹⁰ abaloparatide, the method further comprising:
in step (iii), eluting the beta-Asp¹⁰ abaloparatide from the immobilized stationary phase in the eluant;
in step (iv), detecting the beta-Asp¹⁰ abaloparatide present in the eluant using a detector; and
in step (v), quantifying the beta-Asp¹⁰ abaloparatide present in the sample.

47. The method of claim 46, wherein the sample is from a batch of abaloparatide drug substance.

48. The method of claim 47, wherein the method comprises determining the sample comprises an amount of beta-Asp10 abaloparatide which is ≤0.5% by weight, based on the total weight of the drug substance.

49. The method of claim 48, further comprising qualifying the batch of abaloparatide drug substance as suitable for manufacture of a formulated abaloparatide drug product.

50. The method of claim 48, wherein qualifying the batch comprises creating and approving a batch record, filing a submission with the FDA certifying the batch, or both.

51. The method of claim 48 or 49, further comprising formulating the abaloparatide drug substance into an abaloparatide drug product suitable for subcutaneous administration.

52. The method of claim 47, wherein the method comprises determining the sample comprises an amount of beta-Asp10 abaloparatide which is >0.5% by weight, based on the total weight of the drug substance, and wherein the method comprises conducting further purification of the batch of abaloparatide drug substance, reworking the batch of abaloparatide drug substance, or both.

53. The method of claim 52, further comprising formulating the further purified or reworked batch of abaloparatide drug substance into an abaloparatide drug product suitable for subcutaneous administration.

54. The method of claim 46, wherein the sample is from a batch of formulated abaloparatide drug product.

55. The method of claim 54, wherein the batch of formulated abaloparatide drug product is a batch of an initially prepared, formulated abaloparatide drug product.

56. The method of claim 55, wherein quantifying the beta-Asp¹⁰ abaloparatide present in the sample comprises determining the sample comprises an amount of beta-Asp¹⁰ which is ≤0.5% by weight based on the total weight of abaloparatide present in the batch of the formulated abaloparatide drug product.

57. The method of claim 46, wherein the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

58. The method of claim 57, further comprising determining the suitability for administration to a subject of the batch of stored, formulated abaloparatide drug product, the determining comprising ascertaining that the sample comprises an amount of beta-Asp¹⁰ abaloparatide which is ≤5% by weight based on the total weight of abaloparatide in the batch of stored, formulated abaloparatide drug product.

59. The method of any one of claims 37-58, wherein the sample further comprises cyclo-Asp¹⁰ abaloparatide, the method further comprising:
in step (iii), eluting the cyclo-Asp¹⁰ abaloparatide from the immobilized stationary phase in the eluant;
in step (iv), detecting the cyclo-Asp¹⁰ abaloparatide present in the eluant using a detector; and
in step (v), quantifying the cyclo-Asp¹⁰ abaloparatide present in the sample.

60. The method of claim 59, wherein the sample is from a batch of abaloparatide drug substance.

61. The method of claim 60, wherein the method comprises determining the sample comprises an amount of cyclo-Asp¹⁰ abaloparatide which is ≤0.5% by weight, based on the weight of abaloparatide.

62. The method of claim 59, wherein the sample is from a batch of formulated abaloparatide drug product.

63. The method of claim 62, wherein the batch of formulated abaloparatide drug product is a batch of an initially prepared, formulated abaloparatide drug product.

64. The method of claim 63, wherein quantifying the cyclo-Asp¹⁰ abaloparatide present in the sample comprises determining the sample comprises an amount of cyclo-Asp¹⁰ which is ≤0.5% by weight based on the weight of abaloparatide in the batch of the formulated abaloparatide drug product.

65. The method of claim 59, wherein the sample is from a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

66. The method of claim 65, further comprising determining the suitability for administration to a subject of the batch of stored, formulated abaloparatide drug product, the determining comprising ascertaining that the sample comprises an amount of cyclo-Asp¹⁰ abaloparatide which is ≤3% by weight based on the total weight of abaloparatide in the batch of stored, formulated abaloparatide drug product.

67. A method of providing abaloparatide in a form substantially free of beta-Asp¹⁰ abaloparatide, the method comprising:
(i) providing a sample from a batch of abaloparatide;
(ii) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
(iii) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting components of the sample from the immobilized stationary phase in the eluant;
(iv) detecting abaloparatide and beta-Asp¹⁰ abaloparatide in the sample using a detector;
(v) quantifying the amount of beta-Asp¹⁰ abaloparatide present relative to abaloparatide;
(vi) ascertaining that the amount beta-Asp¹⁰ abaloparatide is not more than 1% by weight based on the weight of abaloparatide; and
(vii) approving release of the batch of abaloparatide.

68. A method of manufacturing an abaloparatide drug product suitable for subcutaneous administration, the method comprising:
(ii) analyzing a sample from a batch of abaloparatide drug product, wherein analyzing comprises:
d) introducing the sample to an ultra-high performance chromatography (UPLC) system comprising a column having an interior for accepting a stationary phase, and an immobilized stationary phase within said interior of the column, wherein the immobilized stationary phase comprises C18-modified particles;
e) flowing the sample through the immobilized stationary phase for a period of time with an eluant comprising a first mobile phase comprising water, formic acid, and heptafluorobutyric acid, and a second mobile phase comprising acetonitrile, formic acid and heptafluorobutyric acid, thereby eluting components of the sample from the immobilized stationary phase in the eluant;
f) detecting and quantifying any isomeric and degradation impurities present in the sample using a detector;
iv) determining the abaloparatide is substantially free of isomeric and degradation impurities; and
v) approving the release of the batch of abaloparatide.

69. The method of claim 68, wherein the batch of abaloparatide drug product is a batch of an initially prepared, formulated abaloparatide drug product.

70. The method of claim 69, wherein determining the batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 1% by weight based on the weight of abaloparatide; and
ascertaining that the amount cyclo-Asp¹⁰ abaloparatide is not more than 0.5% by weight based on the weight of abaloparatide.

71. The method of claim 68, wherein the batch of abaloparatide drug product is a batch of formulated abaloparatide drug product that has been stored for up to 23 months at 2-8° C., has been stored for up to 23 months at 2-8° C. followed by a month at 20-25° C., or has been stored for up to 35 months at 2-8° C. followed by a month at 20-25° C.

72. The method of claim 71, wherein determining the stored batch of formulated abaloparatide drug product is substantially free of isomeric and degradation impurities comprises:
quantifying an amount of beta-Asp¹⁰ abaloparatide present;
quantifying an amount of cyclo-Asp¹⁰ abaloparatide present;
ascertaining that the amount beta-Asp¹⁰ abaloparatide is not more than 5% by weight based on the weight of abaloparatide; and
ascertaining that the amount cyclo-Asp¹⁰ abaloparatide is not more than 3% by weight based on the weight of abaloparatide.


SEQUENCE LISTING (1 sequence)

SEQ ID NO: 1 - protein, 34 aa, synthetic construct
  MOD_RES 15: note = isoleucine
  MOD_RES 29: note = Aib
AVSEHQLLHDKGKSXQDLRRRELLEKLLXKLHTA